(19) United States
(12) Patent Application Publication
(10) Pub. No.: US 2007/0128643 A1
(43) Pub. Date: Jun. 7, 2007

(54) Method for assaying nucleic acid fragment and kit thereof

(76) Inventor: Yang-Chuen Yeh, Pleasanton, CA (US)

Correspondence Address: YANG-CHUEN YEH, 3930 STONERIDGE DRIVE, #3, PLEASANTON, CA, 94588, US

(21) Appl. No.: 11/604,422

(22) Filed: Nov. 28, 2006

(30) Foreign Application Priority Data
Dec. 5, 2005 (TW) 094142769

(51) Int. Cl. C12Q 1/68; C07H 21/04

(52) U.S. Cl. 435/6; 536/25.32

(57) ABSTRACT

The present invention is related to a novel method for assaying high quantity of nucleic acid fragments, by using the single-stranded anchor nucleic acid probe with biotin and this single-stranded anchor nucleic acid hereto form double-stranded nucleic acid with the target nucleic acid fragment. To detect the target fragment does not need electrophoresis or nucleic acid sequencing but needs the antibody against double-stranded nucleic acid or the specific single-stranded reporter nucleic acid labeled with a fluorescent dye. The present invention is also related to a kit of assaying high quantity of nucleic acid fragments. A container (the multiwell plate) coating the avidin family molecule is a tool for connecting with the biotinylated target nucleic acid and subjected to the antibody reaction or fluorescence detection.

### FIELD OF THE INVENTION

The present invention is related to a novel method for assaying high quantity of nucleic acid fragments.

### BACKGROUND OF THE INVENTION

The general traditional method for examining the nucleic acid fragment or product of polymerase chain reaction (PCR), electrophoresis was taken as the core. Utilize the density or size appearing in the electrophoresis of nucleic acid to present the character of the fragment. But if the result of the electrophoresis does not very clear, other methods such as Southern blotting, Northern blotting and DNA sequencing are instead to perform. However, such technologies are very troublesome so that should be operated by specialists and unable to make a large number of sample screening. Bateman found first as far back as 1916 that there was a kind of toxic material in the egg albumin. Boas did not find until 1927 a certain food could prevent the toxicity of albumin. Later a substance that could resist this toxic material in albumin was separated and named biotin. The structure of biotin was deduced and explained in 1936, and this kind of molecule could be synthesized in the laboratory in 1943.

Biotin, commonly referred as Vitamin H or Vitamin B₇, is a trace element that generally exists in the cells. This molecule is quite small, and its molecular weight is 244.3 Dalton. It is a ring-type chemical like urea which has a structure ring of thiophene. It has eight kinds of isomers, but only dextrorotation biotin (d-biotin) exists in the nature with the function of vitamin. Biotin is a material of colorless, needle form, and soluble in the cold water slightly. It is relatively apt to dissolve in alcohol rather than the organic solvent Biotin is fairly heat stable and also not destroyed by acid or base. Biotin has three close partners, avidin, streptavidin and neutravidin. Biotin has quite high affinity with avidin which can form a very strong non-covalent bond (Ka =1015 M⁻¹). The bond forms quickly, and it is difficult to be influenced to dissociate by extreme pH, temperature, organic solvent or detergent once after taking shape. Biotin-avidin complex can be active in the environment of 3 M guanidine HCl (Guanidine Hydrochloride). Until using 8 M guanidine HCl with pH 1.5 or the autoclave, biotin can be released from the complex.

Avidin is a glycoprotein found in the egg white, and also exists in the tissues of birds, reptiles and amphibian. The molecular weight of avidin is 67,000 Dalton. It is a homotetramer that binds four molecules of biotin. There is quite high proportion of saccharide in avidin, almost occupied 10% of the total molecular weight. Its pI is between 10-10.5, and it can dissolve in water and salt solution. Avidin is very stable under quite wide range of temperature and pH.

Neutravidin is the avidin excluding saccharide, and its molecular weight is 60,000 Dalton. It has the neutral pI value, and the lowest non-specific binding with biotin.

Streptavidin is a protein purified from Streptomyces Avidinii, and also can specifically bind to biotin. The molecular weight of streptavidin is 60,000 Dalton. Streptavidin is also a homotetramer that binds to four molecules of biotin, and its affinity with biotin is quite equal to the affinity of avidin with biotin. However biotin-streptavidin complex has the stronger resistance to guanidine HCl than biotin-avidin complex does. In addition, streptavidin contains no saccharide and has the acidic pI 5, which just on the contrary with biotin.

According to the quick and strong binding ability between biotin and avidin (or streptavidin), and plus quite small molecule of biotin, it is very suitable for binding biotin to different biological molecules. Furthermore, the support or enzyme attached to avidin is utilized to separate, purify the biological molecules or for other series detections and examinations. The most common application examples reveal as follows:

a. Immunoblotting:
Biotin is attached to a certain antibody, then utilized streptavidin-horseradish peroxidase (HRP) and the substrate of HRP to proceed with blotting detection.

b. ELISA (Enzyme-linked immunosorbent assay):
Avidin or streptavidin is fixed on the plate, and added the antibody or protein labeled with biotin to carry on the subsequent steps (this assay is also called the sandwich method).

c. Immunoprecipitation (IP/Co-IP)
Avidin attached to bead is utilized to precipitate the protein labeled with biotin or the protein that correlated with the protein labeled with biotin thereof together, then subject to follow-up analysis.

d. Antibody or protein purification:
The antibody or protein attached with biotin is connected with avidin/streptavidin to purify the antibody or protein, and then the biotin molecule is removed from the purified antibody or protein at last step.

e. EMSA (Electrophoretic mobility shift assay, Gel-shift assay):
The specific nucleic acid sequence marked with biotin is mixed with the sample proteins, and then the nucleic acid-protein complex is analyzed by electrophoresis and continued with the detection by streptavidin-HRP and the substance of HRP.

### BRIEF DESCRITION OF THE FIGURES

FIG. 1 shows the detection method mainly in using antibody.

FIG. 2 shows the detection method without using antibody.

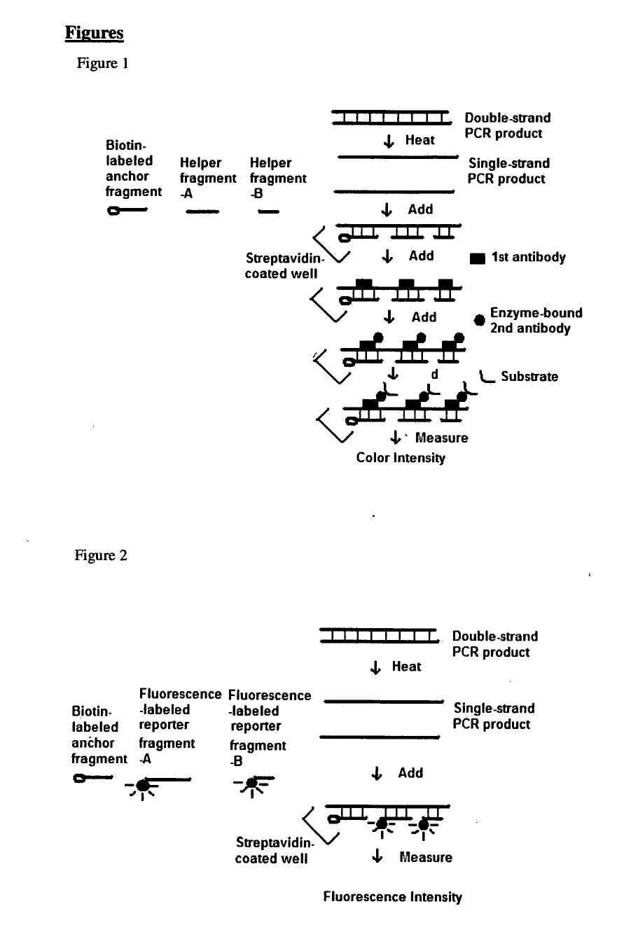

### DESCRIPTION OF THE INVENTION

Traditionally, examining a section of specific nucleic acid in a large amount, either DNA sequencing or utilization of Northern blotting or Southern blotting would be selected. Each process is labor and time-consuming, and the experiment has certain difficulty which is unsuitably for doing a large number of examinations. Examination in a large quantity is especially useful on blood test, even on the detection of bird flu virus or other specific diseases with known nucleic acid sequence.

According to this invention, not only can detect many kinds of nucleic acid fragments (representing many kinds of pathogens) in one kind of sample at the same time, but also can detect the same kind of nucleic acid fragments in many kinds of samples. In addition, it can detect many kinds of nucleic acid fragments in many kinds of samples. Because electrophoresis analysis does not need to make, the whole process can be accelerated. Relative to the prior technologies of assaying the specific nucleic acid fragment, the major advantage of this invention lies in reducing the time to examine by a wide margin. Low cost, fast screening, wide target range and large quantity of samples examined at the same time are all the superior parts of this invention.

This invention utilizes a specific single-stranded nucleic acid probe which can form double-stranded nucleic acid with the fragment of target nucleic acid, in addition to an antibody that can recognize the double-stranded nucleic acid. Therefore, this invention can verify the high quantity of nucleic acid fragments. Though polymerase chain reaction, biotinylation and quantitative analysis of antibody reaction belong to the prior art, all of these three technologies are ingeniously combined by the present invention. The unique combination adds the commercial antibody which recognizes the double-stranded nucleic acid, namely the main concept of the present invention.

The better embodiment of this invention is in denaturation of double-stranded nucleic acid to single-stranded nucleic acid before adding the probe attached with biotin and the specific helper fragment which can recognize the denatured fragment thereof. The purpose of the specific helper fragment is on recognizing the specific target nucleic acid in order to reconstruct part site of the target one. The other purpose is on amplifying the signals expressed by antibody, even through fluorescence or the substance being chromogen after making use of the antibody against double-stranded nucleic acid. This is because the specific helper fragment turns denatured target nucleic acid into part site renatured. The plate is coated tightly with the avidin family molecule, avidin, neutravidin or streptavidin which has high affinity with biotin. Its aim lies in combining the probe attached with biotin and fixing the probe which recognizes the target nucleic acid in the well of plate.

The biotinylated, double-stranded nucleic acid is then fixed in the multiwell plate before adding the primary antibody against double-stranded nucleic acid. Thus, the target nucleic acid will tightly stay on the plate against washing procedures later, so it has the advantage on large quantity of screening. The primary antibody can be attached with a labeling molecule like the fluorescent dye or enzyme, HRP. The secondary antibody can be also attached with a fluorescent dye or enzyme to present the target There are two kinds of commonly used substrate of HRP: 4-chlorine-1-naphthol and TMB (3,3,5,5-tetramethylbenzidine).

Another better embodiment of this invention is in connecting the fluorescent or chemiluminescent material directly with the specific reporter fragment. The biotinylated probe is fixed in the plate coating with the avidin family molecule after combining the target nucleic acid. Next the specific reporter nucleic acid with fluorescein is added for detection. Then measure absorbance directly to confirm the existence of target one thereof.

Nucleic Acid Probe Design

The length of anchor nucleic acid probe having biotin is composed of 10-60 nucleotides. The length of 0-10 helper/reporter nucleic acid probes is composed of 10-60 nucleotides. These helper/reporter probes form double-stranded nucleic acid with the same strand of target nucleic acid.

The present invention is related to a method of screening a nucleic acid fragment in large quantity comprising:
(a). hybridizing an anchor nucleic acid probe attached with biotin and a single-stranded target nucleic acid denatured by heating;
(b). i. adding the single-stranded specific helper nucleic acid fragment; or
   ii. adding the single-stranded specific reporter nucleic acid fragment with fluorescein;
(c). adding double-stranded mixture into an avidin family molecule; and
(d). adding an antibody against double-stranded nucleic acid to confirm target nucleic acid after double-stranded mixture fixed in the plate by combination of the avidin family molecule and biotin.

According to the method of the present invention, the target nucleic acid fragment comes from polymerase chain reaction (PCR) products or other nucleic acid sources. The anchor nucleic acid probe mentioned above is a single-stranded DNA. The sequence of specific helper/reporter nucleic acid fragment complements with the sequence of target nucleic acid to enhance the screening signals.

The avidin family molecule of the present invention means avidin, neutravidin or streptavidin. The preferred embodiment is streptavidin. The antibody of the present invention includes the primary antibody or secondary antibody. The primary antibody links a fluorescent dye; however, the secondary antibody links a chemiluminent enzyme (such as HRP) which can be used to generate the signals.

The present invention is also related to a kit of screening a nucleic acid fragment in large quantity comprising:
(a). an anchor nucleic acid probe attached with biotin
(b). i. a single-stranded specific helper nucleic acid fragment; or
   ii. the single-stranded specific reporter nucleic acid fragment attached with fluorescein.
(c). a container coated with an avidin family molecule; and
(d). a primary antibody against double-stranded nucleic acid.

According to the kit of the present invention, further comprises an instruction to describe contents and protocols. The nucleic acid fragment comes from PCR products or other nucleic acid sources. The kit of the present invention requires the anchor nucleic acid probe having biotin. This probe is a single-stranded DNA. The sequence of specific helper/reporter nucleic acid fragment complements with the sequence of target nucleic acid to enhance the screening signals. The avidin family molecule means avidin, neutravidin or streptavidin. The preferred embodiment is streptavidin. The purpose of the container is on fixing anchor nucleic acid probe attached with biotin and therefore fixing double-stranded nucleic acid indirectly. The primary antibody of the kit links a fluorescent dye; however, the secondary antibody links a chemiluminent enzyme (such as HRP) which can be used to generate the signals.

The application of this present invention is on screening large quantity of nucleic acid fragments which come from PCR products or other nucleic acid sources. The present invention also can apply to the screening of nucleic acid fragments which come from blood samples but not limit to this.

The examples below are non-limiting, and are merely representative of various aspects and features of the present invention.

### EXAMPLE

### Example 1

1. The DNA fragments or PCR products were heated at 95-100° C. for 1 to 5 minutes, and cooled down on ice to maintain the single-stranded status. Then the samples were transferred into a multiwell plate containing streptavidin.

2. The anchor nucleic acid probe linked with biotin and the specific helper nucleic acid fragment were added into the multiwell plate.

3. The mixture was shaken evenly at the room temperature for 30-90 minutes.

4. The mixture was removed after shaking, and added PBS buffer (5×PBS buffer: NaCl 0.13 M, NaH₂PO₄ 0.101 M in 1 L water, pH 7.0) to wash the plate for 5-30 minutes at the room temperature.

5. The primary antibody which against double-stranded DNA was added after washing, and continued with shaking for 30-120 minutes at the room temperature.

6. The mixture was removed after shaking, and added PBS buffer to wash the plate for 5-60 minutes at the room temperature.

7. The secondary antibody attached with HRP was added, and shook at room temperature for 30-120 minutes.

8. The mixture was removed after shaking, and added PBS buffer to wash the plate for 5-60 minutes at the room temperature.

9. After adding the substrate of HRP, measured the color reaction to determine the DNA fragment.

### Example 2

1. The DNA fragments or PCR products were heated at 95-100°C. for 1-5 minutes, and cooled down on ice to maintain the single-stranded status. Then the samples were transferred into a multiwell plate containing streptavidin.

2. The anchor nucleic acid probe linked with biotin and the specific reporter nucleic acid attached with a fluorescent dye were added into the multiwell plate.

3. The mixture was shaken evenly at the room temperature for 30-90 minutes.

4. The mixture was removed after shaking, and added PBS buffer to wash the plate for 5-30 minutes at the room temperature.

5. The fluorescence was measured to determine the DNA fragment.

One skilled in the art readily appreciates that the present invention is well adapted to carry out the objects and obtain the ends and advantages mentioned, as well as those inherent therein. The processes and methods for producing them are representative of preferred embodiments, are exemplary, and are not intended as limitations on the scope of the invention. Modifications therein and other uses will occur to those skilled in the arL These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of screening a nucleic acid fragment in large quantity comprising:
(a). hybridizing an anchor nucleic acid probe attached with biotin and a single-stranded target nucleic acid denatured by heating;
(b). i. adding the single-stranded specific helper nucleic acid fragment; or
ii. adding the single-stranded specific reporter nucleic acid fragment with fluorescein;
(c). adding double-stranded mixture into an avidin family molecule; and
(d). adding antibody against double-stranded nucleic acid to confirm target nucleic acid after double-stranded mixture fixed in the plate by combination of the avidin family molecule and biotin.

2. According to the method of claim 1, wherein the target nucleic acid fragment comes from polymerase chain reaction (PCR) products or other nucleic acid sources.

3. According to the method of the claim 2, wherein the target nucleic acid fragment is DNA.

4. According to the method of the claim 1, wherein the anchor nucleic acid probe is DNA, and attached with biotin.

5. According to the method of claim 1, wherein the specific helper or reporter nucleic acid fragment having the sequence complements with the sequence of target nucleic acid.

6. According to the method of claim 1, wherein the specific helper nucleic acid fragment having the purpose to enhance the screening signals.

7. According to the method of claim 1, wherein the avidin family molecule is avidin, neutravidin or strepavidin.

8. According to the method of claim 7, wherein the avidin family molecule is strepavidin.

9. According to the method of claim 1, wherein the antibody is the primary antibody and a secondary antibody can be added.

10. According to the method of claim 9, wherein the primary antibody can be attached with a fluorescent dye.

11. According to the method of claim 9, wherein the secondary antibody can be attached with a chemiluminent enzyme.

12. A kit of screening a nucleic acid fragment in large quantity comprising:
(a). an anchor nucleic acid probe attached with biotin;
(b). i. a single-stranded specific helper nucleic acid fragment; or
ii. the single-stranded specific reporter nucleic acid fragment attached with fluorescein.
(c). a container coated with an avidin family molecule; and
(d). a primary antibody against double-stranded nucleic acid.

13. According to the kit of claim 12, further comprises the secondary antibody having a recognition molecule.

14. According to the kit of the claim 12, further comprises an instruction to describe contents and protocols.

15. According to the kit of the claim 12, wherein the nucleic acid fragment comes from PCR products or other nucleic acid sources.

16. According to the kit of the claim 15, wherein the nucleic acid fragment is DNA.

17. According to the kit of the claim 12, wherein the avidin family molecule is avidin, neutravidin or strepavidin.

18. According to the kit of claim 17, wherein the avidin family molecule is strepavidin.

19. According to the kit of the claim 12, wherein the container is on fixing nucleic acid probe attached with biotin.

20. According to the kit of the claim 12, wherein the primary antibody can be attached with a fluorescent dye directly.

21. According to the kit of the claim 13, wherein the secondary antibody can be attached with a chemiluminent enzyme.

22. According to the kit of the claim 12, wherein the anchor nucleic acid probe is attached with biotin.

23. According to the kit of the claim 22, wherein the nucleic acid probe is DNA.

24. According to the kit of the claim 12, wherein the specific helper or reporter nucleic acid having the sequence complements with the sequence of target nucleic acid.

25. According to the kit of the claim 12, wherein the specific helper nucleic acid fragment having the purpose to enhance the screening signals.
